(19) United States
(12) Patent Application Publication
(10) Pub. No.: US 2005/0277584 A1
(43) Pub. Date: Dec. 15, 2005

(54) Pharmaceutical compositions comprising cyclosporins

(75) Inventors: Walter L. Tien, Irvine, CA (US); Richard Graham, Irvine, CA (US); James N. Chang, Newport Beach, CA (US)

Correspondence Address: ALLERGAN, INC., LEGAL DEPARTMENT, 2525 DUPONT DRIVE, T2-7H, IRVINE, CA, 92612-1599, US

(73) Assignee: ALLERGAN, INC., Irvine, CA (US)

(21) Appl. No.: 10/865,638

(22) Filed: Jun. 9, 2004

(51) Int. Cl. A61K 38/13; A61K 31/355

(52) U.S. Cl. 514/11; 514/458

(57) ABSTRACT

A liquid comprising a therapeutically effective concentration of a cyclosporin and a vitamin E tocopherol polyethylene glycol succinate, wherein said liquid is an aqueous solution, and wherein no hydrophilic organic solvent is present at a concentration greater than half of that of the cyclosporin is also disclosed herein. A composition comprising a therapeutically effective concentration of cyclosporin A and an effective amount of a vitamin E tocopherol polyethylene glycol succinate, wherein said composition is an aqueous liquid solution which is intended for ophthalmic use, and wherein no hydrophilic organic solvent is present at a mass concentration greater than or equal to that of the cyclosporin, is disclosed herein. A composition comprising a therapeutically effective concentration of cyclosporin A and an effective amount of a vitamin E tocopherol polyethylene glycol succinate, wherein said composition is an aqueous liquid solution which is intended for parenteral use, and wherein no hydrophilic organic solvent is present at a mass concentration greater than or equal to that of the cyclosporin, is disclosed herein. Methods of treating diseases or conditions using said compositions, and medicaments related thereto, are also disclosed herein.

### FIELD OF THE INVENTION

The present invention relates to pharmaceutical compositions. In particular, the present invention relates to compositions comprising cyclosporins.

### BACKGROUND OF THE INVENTION

Description of the Related Art

Dry eye disease is a general term for a variety of conditions characterized by abnormalities in the tear film, which affects three million people in the United States alone. Dry eye is characterized by symptoms such as a sandy-gritty feeling in the eye, burning, irritation, or a foreign-body sensation that worsens during the day. Patients suffering from dry eye disease complain of mild to severe symptoms, and those with severe symptoms may experience constant and disabling eye irritation, and develop ocular surface epithelial disease and sight-threatening sterile or microbial corneal ulceration.

Cyclosporins are a group of nonpolar cyclic oligopeptides with immunosuppressant, anti-inflammatory, and anti-parasitic properties. Cyclosporin A is a cyclosporin which is marketed in a topical ophthalmic emulsion formulation for the treatment of dry eye by Allergan, Inc. under the tradename Restasis®. The insolubility of cyclosporins in water is an ongoing problem in the formulation of these compounds.

WO0008085 discloses “a composition for oral administration comprising (i) an immunosuppressant, e.g. cyclosporin, (ii) tocopherol (Vitamin E), tocotrienol or a derivative thereof, (iii) a short chain phospholipid, and (iv) a non-ionic surfactant”, and claims that “composition of the invention can provide for good solubility of the immunosuppressant, e.g. cyclosporin, in an excipient mixture as well as good dispersibility when placed in an aqueous environment”.

U.S. Pat. No. 5,798,333 discloses “pharmaceutical compositions which enable high concentrations of a cyclosporin and are water-soluble, such that the compositions will dissolve in aqueous media without precipitation of the cyclosporin. The compositions comprise a cyclosporin dissolved in tocophersolan and a hydrophilic organic solvent, preferably propylene glycol.” The patent further discloses that “the solvent selected should be an efficient solvent for cyclosporin, and also a solvent for tocophersolan.

Preferred organic solvents meeting these criteria include but are not necessarily limited to propylene glycol and various monoalcohols, including ethanol, benzyl alcohol, hexanol, and phenethyl alcohol.

Most preferred is propylene glycol because it has low toxicity and low volatility in addition to being an efficient solvent for cyclosporin.

The amount of propylene glycol needed to provide a stable solution of cyclosporin and tocophersolan is about 1 g per g of cyclosporin. A suitable solution preconcentrate will thus consist of 1 part cyclosporin, 7.5 parts tocophersolan and 1 part propylene glycol.”

U.S. Patent Application Publication No. 20030108626, published on Jun. 12, 2003, and filed on Nov. 1, 2001, discloses “a method and composition for treating a dry eye condition by topically applying to the eye surfaces an emulsion . . . Includable in the mixture is a non-soluble therapeutic agent, such as cyclosporin which is effective against an eye disease and is delivered to the eye by the film”.

### BRIEF DESCRIPTION OF THE INVENTION

A liquid comprising a therapeutically effective concentration of a cyclosporin and a vitamin E tocopherol polyethylene glycol succinate, wherein said liquid is an aqueous solution, and wherein no hydrophilic organic solvent is present at a concentration greater than half of that of the cyclosporin is also disclosed herein.

A composition comprising a therapeutically effective concentration of cyclosporin A and an effective amount of a vitamin E tocopherol polyethylene glycol succinate, wherein said composition is an aqueous liquid solution which is intended for ophthalmic use, and wherein no hydrophilic organic solvent is present at a mass concentration greater than or equal to that of the cyclosporin, is disclosed herein.

A composition comprising a therapeutically effective concentration of cyclosporin A and an effective amount of a vitamin E tocopherol polyethylene glycol succinate, wherein said composition is an aqueous liquid solution which is intended for parenteral use, and wherein no hydrophilic organic solvent is present at a mass concentration greater than or equal to that of the cyclosporin, is disclosed herein.

Methods of treating diseases or conditions using said compositions, and medicaments related thereto, are also disclosed herein.

### DETAILED DESCRIPTION OF THE INVENTION

The compositions disclosed herein are aqueous liquid solutions according to the meaning generally understood in the art.

The term “cyclosporin” refers to any cyclosporin compounds known in the art including cyclosporin A, cyclosporin B, cyclosporin C, cyclosporin D, and cyclosporin G. In certain compositions, the cyclosporin is cyclosporin A.

The term “vitamin E tocopherol polyethylene glycol succinate” refers to an ester compound or a mixture of compounds derived from succinic acid, polyethylene glycol, and tocopherol. The compounds are diesters of succinic acid, where the two ester linkages occur to a phenolic hydroxyl group of the tocopherol and a hydroxyl group of polyethylene glycol. Polyethylene glycol is HO(CH₂CH₂O)ₙH, otherwise known as polyethylene oxide. The term tocopherol refers to a naturally occurring form of vitamin E, and may refer to a single compound or a mixture. Examples of tocopherols include α-tocopherol, dl-α-tocopherol, β-tocopherol, γ-tocopherol, and δ-tocopherol. Polyethylene glycol is the well known polymer of ethylene glycol. One useful tocopherol which is conveniently obtained commercially is sold by Eastman Chemical as Vitamin E TPGS NF. The US Pharmacopeia has designated tocophersolan as the name for Vitamin E TPGS NF.

The term “hydrophilic organic solvent” refers to an organic compound which is an efficient solvent for cyclosporin, and also a solvent for tocophersolan. Examples of hydrophilic organic solvents include propylene glycol and water-soluble monoalcohols, including ethanol, benzyl alcohol, hexanol, and phenethyl alcohol. In certain compositions, no hydrophilic organic solvent is present at a mass concentration greater than or equal to that of the cyclosporin. In other words, there is a greater mass of the cyclosporin than any hydrophilic solvent which may be present in the solution. In other compositions, no hydrophilic organic solvent is present at a mass concentration greater than half of that of the cyclosporin.

Certain compositions contain essentially no hydrophilic organic solvent.

A therapeutically effective concentration of cyclosporin is a concentration useful to observe a therapeutic effect as compared to a placebo composition having the same composition sans cyclosporin, and can be determined by a person of ordinary skill in the art without undue experimentation. While not intending to limit the scope of the invention in any way, the water solubility of cyclosporin A is 0.0007% by weight, so the use of vitamin E tocopherol polyethylene glycol succinate in the composition is often useful when the cyclosporin concentration is 0.001% or greater. In other embodiments, the concentration of cyclosporin is greater than 0.01%. In other embodiments, the concentration of cyclosporin is greater than 0.02%. In other embodiments, the concentration of cyclosporin is at least 0.05%. For the treatment of dry eye disease, a cyclosporin concentration of less than or equal to 1% is often adequate. In other words, in certain compositions, the concentration of the cyclosporin is at or below 1%. In other embodiments, the concentration of cyclosporin is at or below 0.2%. In other embodiments, the concentration of cyclosporin is at or below 0.15%. In other embodiments, the concentration of cyclosporin is about 0.05%. In other embodiments, the concentration of cyclosporin is about 0.1%.

An effective amount of vitamin E tocopherol polyethylene glycol succinate is the amount useful to enhance solubility of the cyclosporin, and will depend upon the amount and kind of cyclosporin used, as well as what other excipients may be present in the composition. While not intending to limit the scope of the invention in any way, in many cases a vitamin E tocopherol polyethylene glycol succinate concentration of at least 0.5% is useful. In other cases a vitamin E tocopherol polyethylene glycol succinate concentration of at least 1% is useful. In certain cases, the vitamin E tocopherol polyethylene glycol succinate concentration may be less than or equal to 5%. Often, a concentration of vitamin E tocopherol polyethylene glycol succinate which is at least 8 times the concentration of the cyclosporin is useful. In other cases, the concentration of vitamin E tocopherol polyethylene glycol succinate and the concentration of cyclosporin have a ratio of 10. In other cases the ratio may be even greater. In other words, there will be 10 mg of vitamin E tocopherol polyethylene glycol succinate for every 1 mg of cyclosporin in a given amount of solution, or in certain instances there may be even more than 10 mg of vitamin E tocopherol polyethylene glycol succinate for every mg of cyclosporin present in a given amount of solution. In certain compositions, the concentration of vitamin E tocopherol polyethylene glycol succinate is no more than 15 times the concentration of the cyclosporin.

A liquid which is intended for ophthalmic use is formulated such that it can be administered topically to the eye. The comfort should be maximized as much as possible, although sometimes formulation considerations may necessitate less than optimal comfort. In the case that comfort cannot be maximized, the liquid should be formulated such that the liquid is tolerable to the patient for topical ophthalmic use. Additionally, an ophthalmically acceptable liquid may be packaged for single use, or contain a preservative to prevent contamination over multiple uses.

As is known in the art, buffers are commonly used to adjust the pH to a desirable range for ophthalmic use. Generally, a pH of around 5-8 is desired, however, this may need to be adjusted due to considerations such as the stability or solubility of the therapeutically active agent or other excipients. Many buffers including salts of inorganic acids such as phosphate, borate, and sulfate are known.

Another commonly used excipient in ophthalmic compositions is a viscosity-enhancing, or a thickening agent. Thickening agents may be used for a variety of reasons, ranging from improving the form of the formulation for convenient administration to improving the contact with the eye to improve bioavailability. The thickening agent may comprise a polymer containing hydrophilic groups such as monosaccharides, polysaccharides, ethylene oxide groups, hydroxyl groups, carboxylic acids or other charged functional groups. While not intending to limit the scope of the invention, some examples of useful thickening agents are sodium carboxymethylcellulose, hydroxypropylmethylcellulose, povidone, polyvinyl alcohol, and polyethylene glycol.

In ophthalmic solutions, tonicity agents may be used to adjust the composition of the formulation to the desired isotonic range. Tonicity agents are well known in the art and some examples include glycerin, mannitol, sorbitol, sodium chloride, and other electrolytes.

Preservatives may be used to prevent bacterial contamination in multiple-use ophthalmic preparations. Preservatives are well known in the art, and, while not intending to be limiting, examples include polyhexamethylenebiguanidine (PHMB), benzalkonium chloride (BAK), stabilized oxychloro complexes (otherwise known as Purite®), phenylmercuric acetate, chlorobutanol, benzyl alcohol, parabens, and thimerosal are examples of useful preservatives.

In ophthalmic compositions, a chelating agent may be used to enhance preservative effectiveness. Suitable chelating agents are those known in the art, and, while not intending to be limiting, edetate (EDTA) salts like edetate disodium, edetate calcium disodium, edetate sodium, edetate trisodium, and edetate dipotassium are examples of useful chelating agents.

The compositions disclosed herein are useful in the treatment of dry eye disease, and in the preparation of medicaments for the treatment of dry eye disease. However, certain compositions disclosed herein are also useful for the treatment or prevention of other conditions or diseases which are related to immune response, inflammatory response, or parasitic or other infection.

The compositions disclosed herein are also useful for parenteral administration of a cyclosporin. A composition which is formulated for parenteral use is a composition which is formulated with the intention of administering the composition parenterally. Parenteral administration is generally characterized by injection, either subcutaneously, intramuscularly or intravenously. While not intending to limit the scope of the invention in any way, in addition to vitamin E tocopherol polyethylene glycol succinate, suitable excipients are, for example, saline, dextrose, buffering agents, and the like.

The best mode of making and using the present invention are described in the following examples. These examples are given only to provide direction and guidance in how to make and use the invention, and are not intended to limit the scope of the invention in any way.

### EXAMPLE 1

Formulations 1-4 in Table 1 below were prepared according to the following procedures.
Formulation 1 was prepared by adding 1 mg of cyclosporin into 100 μL of a 10% tocophersolan stock solution and then mixed until dissolved. To this clear solution is slowly added 890 μL of water to yield a clear solution containing 0.1% cyclosporin and 1% tocopehersolan.
Formulations 2 was prepared by adding 1 mg of cyclosporin into 10 μL polysorbate 80 and 10 μL of propylene glycol, and then mixed until dissolved. To this clear solution is slowly added 980 μL of water to yield a turbid solution containing 0.1% cyclosporin and 1% polysorabte 80 and 1% propylene glycol.
Formulation 3 was prepared by adding 1 mg of cyclosporin into 100 μL of a 10% polyoxy-40-stearate stock solution and then mixed until dissolved. To this clear solution is slowly added 890 μL of water to yield a turbid solution containing 0.1% cyclosporin and 1% polyoxy-40-stearate. This cloudy solution remained turbid even with the addition of 10 μL of propylene glycol.

Formulations 4 was prepared by adding 1 mg of cyclosporin into 10 μL polyethylene glycol 400 (PEG 400) and 10 μL of propylene glycol, and then mixed until dissolved. To this clear solution is slowly added 980 μL of water to yield a turbid solution containing 0.1 % cyclosporin and 1% PEG 400 and 1% propylene glycol.

TABLE 1

| | Cyclosporin | Surfactant | | |
|---|---|---|---|---|
| Formu- | concentration | concentration | Type of | Physical |
| lation | (% w/v) | (% w/v) | Surfactant | Appearance |
| 1 | 0.1 | 1.0 | Tocophersolan | Clear |
| 2 | 0.1 | 1.0 | Polysorbate 80 | Precipitation |
| 3 | 0.1 | 1.0 | Polyoxyl-40- | Precipitation |
| | | | stearate | |
| 4 | 0.1 | 1.0 | PEG400 | Precipitation |

While not intending to limit the scope of the invention in any way, or to be bound in any way by theory, Formulation 1, which uses a vitamin E tocopherol polyethylene glycol succinate is a clear solution, while the other formulations are not. In contrast to the formulation 1, the other formulations required propylene glycol as indicated in the procedures above. In addition to being a superior surfactant, vitamin E tocopherol polyethylene glycol succinates are generally regarded in the art to have an excellent toxicology profile, and be generally less irritating than most other surfactants.

### EXAMPLE 2

A preserved cyclosporin solution appropriate for ophthalmic use (composition in Table 2) was prepared according to the following procedure. Cyclosporin (0.05 g) is dissolved in 5 mL of a 10% tocophersolan, 0.6% boric acid at pH 7.4 stock solution and then mixed until dissolved. To this clear solution was slowly added approximately 90 mL of a boric acid solution (boric acid stock solution; 0.6% boric acid adjusted to pH 7.4 with sodium hydroxide). The pH of this clear solution was confirmed to be 7.4, and then 0.455 mL of a Purite® stock solution (2.2%) was added. The clear solution was q.s. to 100 mL with the boric acid stock solution, and then sterile filtered.

TABLE 2

| | Amount or |
|---|---|
| Ingredient | concentration (% w/v) |
| Cyclosporin A | 0.05 |
| Tocophersolan | 0.5 |
| Boric Acid | 0.6 |
| Purite ® (stabilized oxychloro complex) | 0.01 |
| Sodium Hydroxide | pH adjusted to 7.3-7.5 |

### EXAMPLE 3

Dry eye is treated using the composition of Example 2. Relief of symptoms is experienced.

### EXAMPLE 4

The composition of Example 2 is administered by intravenous injection to a patient receiving a kidney transplant. Rejection of the kidney by the patient is suppressed.

1. A liquid comprising a therapeutically effective concentration of a cyclosporin and a vitamin E tocopherol polyethylene glycol succinate, wherein said liquid is an aqueous solution, and wherein no hydrophilic organic solvent is present at a mass concentration greater than half of that of the cyclosporin.

2. The liquid of claim 1 which contains essentially no hydrophilic organic solvent.

3. The liquid of claim 1 wherein the vitamin E tocopherol polyethylene glycol succinate is present at a concentration which is at least 8 times that of the cyclosporin, and wherein the vitamin E tocopherol polyethylene glycol succinate is present at a concentration which is no more than 15 times that of the cyclosporin.

4. The liquid of claim 1 wherein at least 10 mg of the vitamin E tocopherol polyethylene glycol succinate is present for every mg of the cyclosporin present in said solution.

5. The liquid of claim 1 wherein the vitamin E tocopherol polyethylene glycol succinate and the cyclosporin have a concentration ratio of about 10 to 1.

6. The liquid of claim 1 wherein vitamin E tocopherol polyethylene glycol succinate is present at a concentration that is no less than 0.5%, and wherein the vitamin E tocopherol polyethylene glycol succinate is present at a concentration that is no greater than 5%.

7. The liquid of claim 3 comprising about 0.1% cyclosporin A and about 1% vitamin E tocopherol polyethylene glycol succinate.

8. The liquid of claim 2 comprising cyclosporin A, wherein cyclosporin A is present at a concentration of at least 0.01%, and wherein cyclosporin A is not present at a concentration which is greater than 0.2%.

9. The liquid of claim 1 consisting essentially of a therapeutically effective concentration of cyclosporin A, an effective amount of a vitamin E tocopherol polyethylene glycol succinate, water, and an effective amount of one or any combination of excipients selected from the group consisting of buffers, thickening agents, tonicity agents, preservatives, and chelating agents.

10. A composition comprising a therapeutically effective concentration of cyclosporin A and an effective amount of a vitamin E tocopherol polyethylene glycol succinate, wherein said composition, is an aqueous liquid solution which is intended for ophthalmic use, and wherein no hydrophilic organic solvent is present at a mass concentration greater than or equal to that of cyclosporin A.

11. The composition of claim 10 wherein cyclosporin A is present at a concentration at or below 1%.

12. The composition of claim 10 wherein cyclosporin A is present at a concentration which is at least 0.02% and wherein cyclosporin A is present at a concentration which is less than or equal to 0.15%.

13. The composition of claim 12 comprising about 0.05% cyclosporin A.

14. The composition of claim 12 comprising about 0.1% cyclosporin A.

15. The composition of claim 14 comprising about 0.1% cyclosporin A and about 1% vitamin E tocopherol polyethylene glycol succinate.

16. A method of treating dry eye disease comprising administering to a patient an effective amount of a solution comprising cyclosporin A and an effective amount of a vitamin E tocopherol polyethylene glycol succinate, and wherein no hydrophilic organic solvent is present at a mass concentration greater than or equal to that of the cyclosporin in said solution.

17. The method of claim 16 comprising at least 0.001% cyclosporin A and wherein cyclosporin A is present at a concentration which is less than or equal to 1%.

18. The method of claim 16 wherein said solution comprises about 0.1% cyclosporin A and about 1% vitamin E tocopherol polyethylene glycol succinate.

19. The liquid of claim 1 which is intended for parenteral use.

20. The liquid of claim 1 which is intended for ophthalmic use.

21. The liquid of claim 20 comprising about 0.1% cyclosporin A and about 1% vitamin E tocopherol polyethylene glycol succinate.

22. A composition comprising a therapeutically effective concentration of cyclosporin A and an effective amount of a vitamin E tocopherol polyethylene glycol succinate, wherein said composition is an aqueous liquid solution which is intended for parenteral use, and wherein no hydrophilic organic solvent is present at a mass concentration greater than or equal to that of cyclosporin A.

23. The composition of claim 22 wherein cyclosporin A is present at a concentration which is at least 0.02% and wherein cyclosporin A is present at a concentration which is less than or equal to 0.15%.

24. The composition of claim 23 comprising about 0.1% cyclosporin A and about 1% vitamin E tocopherol polyethylene glycol succinate.
